(19) United States
(12) Patent Application Publication
(10) Pub. No.: US 2010/0004493 A1
(43) Pub. Date: Jan. 7, 2010

(54) Integrated Process

(76) Inventors: John R. Porter, Friendswood, TX (US); Marcus L. H. Van Nuland, Rijen (NL); Joseph M. Cheben, Doba (QA); Ronald L. DeMartino, Kingwood, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/166,678

(22) Filed: Jul. 2, 2008

(51) Int. Cl. C07C 5/22

(52) U.S. Cl. 585/303

(57) ABSTRACT

The invention relates to the integration of plural processes around a single device. The plural processes are characterized by having at least two separate and distinct feedstreams, two separate and distinct products, or a combination thereof.

### FIELD OF THE INVENTION

The present invention relates to an integrated process, in more particular embodiments to an integrated disproportionation and isomerization process, and in even more particularly embodiments to an integrated disproportionation and isomerization process for producing toluene and xylene.

### BACKGROUND OF THE INVENTION

Generally it is preferable to integrate chemical processes in such a way as to maximize energy efficiency, such as by minimizing the overall loss of energy which may be required for heating and cooling of process streams. However, since in process applications energy can be expended in different forms, a straightforward determination of maximum energy efficiency is not necessarily possible. Accordingly, incremental improvements are constantly sought.

Integration of a reaction systems per se are known. For instance, integration of two different catalyst systems, e.g., one having gravity-flowing catalyst particles with one having a fixed-bed system is described in U.S. Pat. No. 3,864,240.

In U.S. Pat. No. 4,911,822, a combined process of catalytically hydroreforming a heavy naphtha in at least one reaction zone and catalytically hydroisomerizing a light naptha in at least one reaction zone is disclosed, with the invention characterized in that the hydrogen produced in the hydroreforming unit is used to isomerize the light naphtha, the obtained reformate and isomerate being fractionated preferably together in the same stabilization column.

U.S. Pat. No. 5,227,554 teaches that at least one or both of the effluent streams from the first and second isomerization zones are conveyed to a gas-liquid separator which separates a hydrogen-rich recycle stream. At least a portion of the hydrogen-rich recycle stream is conveyed to one hydrocarbon feed stream and at least a portion of the hydrogen-rich recycle stream is conveyed to another hydrocarbon feed stream whereby the hydrogen recycle stream is shared during both isomerization reactions. The product stream is conveyed to a shared stabilizer which removes the gaseous and volatile components.

U.S. 20020004533 A1 teaches heating a hydrogen recycle stream from a hydrotreater using the energy from a first shift reaction, thereby eliminating the need for a fired heater to heat the hydrogen recycle stream.

The present inventors have discovered a method of improving the energy efficiency of a chemical process, and reducing the equipment and associated capital cost of the process installation, by integrating around a single device.

### SUMMARY OF THE INVENTION

The invention relates to the integration of plural processes around a single device. The plural processes are characterized by having at least two separate and distinct feedstreams, two separate and distinct products, or a combination thereof.

Thus, in an embodiment, there is an integrated process for converting one or more feeds into multiple products, the process comprising a first process A for producing a product PA, and a second process B, different from A, for producing a product PB, which may be the same or different from PA, each separate process, A and B, having a common intermediate process step, wherein a single device is provided for conducting the common intermediate process step.

In a preferred embodiment, an intermediate product is produced in or by the single device, and in embodiments the intermediate product is a common intermediate to both process A and process B.

In other embodiments, which may include a more preferred embodiment of the preferred embodiment set forth immediately above, the single device is a compressor. Thus, in more preferred embodiments, a single compressor is used to compress an intermediate product of Process A and an intermediate product of Process B, and in a yet still more preferred process the intermediate product is a common intermediate product of both Process A and Process B.

In a preferred embodiment, which may be an embodiment of any of the above mentioned embodiments, preferred, more preferred, or otherwise, process A is a disproportionation process. In still more preferred embodiments, the feed of process A is toluene.

In another preferred embodiment, which may also be an embodiment of any of the above mentioned embodiments, preferred, more preferred, or otherwise, process B is an isomerization process. In still more preferred embodiments, the feed of process B may be xylene.

In yet still another preferred embodiment, which again may also be an embodiment of any of the above mentioned embodiments, preferred, more preferred, or otherwise, the respective intermediates in both processes may comprise hydrogen.

In a very preferred embodiment, Process A is a disproportionation process, preferably comprising the disproportionation of toluene, and Process B is an isomerization process, preferably the isomerization of xylene, the device is a compressor, and the intermediate product, in common with both Process A and Process B, is hydrogen.

In other preferred embodiments there may be yet a third process, C, integrated around the same device as first and second Processes A, B. Other processes may also be integrated therewith and also there is the embodiment of having more than one device integrated so that more than one processes may use yet a second device.

In still another embodiment, at least one of processes (e.g., A, B, C (if present) and so forth) is a process for reforming.

Also contemplated as being an aspect of the invention is an apparatus for carrying out the invention as described in the above embodiments, particularly an apparatus which is also integrated with a chemical plant and/or oil refinery.

It is an object of the invention to provide a process characterized by providing a single device for conducting the common intermediate process step, whereby the energy efficiency of the overall, integrated process is improved in comparison to the energy efficiency of processes A and B carried out separately.

It is yet still another object of the invention to integrate two separate reactions, each requiring a compressor, around a single, common, compressor, such as a recycle gas compressor.

It is moreover another object of the invention to integrate a disproportionation reaction and an isomerization reaction around a single, common, compressor.

These and other objects, features, and advantages will become apparent as reference is made to the following detailed description, preferred embodiments, examples, and appended claims

### BRIEF DESCRIPTION OF THE DRAWINGS

In the accompanying drawings, like reference numerals are used to denote like parts throughout the several views.

FIG. 1 presents a diagrammatic process flow diagram of an integrated process according to an embodiment of the invention.

FIG. 2 presents a diagrammatic process flow diagram of an integrated process according to another embodiment of the invention.

### DETAILED DESCRIPTION

The invention relates to the integration of plural processes around a single device, such as a recycle gas compressor. The plural processes are characterized by having at least two separate and distinct feedstreams, two separate and distinct products, or a combination thereof. By “separate” is meant that the physical conduits are different and by “distinct” means that the fluids conveyed in said conduits are different. According to preferred embodiments of the invention, the chemical reaction occurring in process A is different from that occurring in process B, e.g., in these preferred embodiments the invention is not concerned with integration of two isomerization reactions, two disproportionation reactions, or two reforming processes.

Thus, as an example of an embodiment of the invention, one of the processes (process A) may be toluene disproportionation, so that the feedstream is toluene and the effluent from the reactor is benzene and xylene, and one of the processes (process B) may be xylene isomerization, so that the feedstream is xylene (which may be part of the effluent of process A) having a first distribution of isomers of xylene and the effluent is xylene having a second distribution of isomers different from the first distribution of isomers of xylene. These processes have, in common, the same compressor for hydrogen gas recycle.

In a more general embodiment, there is provided an integrated process for converting one or more feeds into multiple products, the process comprising a first process A for producing a product PA, and a second process B, different from A, for producing a product PB, which may be the same or different from PA, each separate process, A and B, having a common intermediate process step, wherein a single device is provided for conducting the common intermediate process step. Additional embodiments have been set forth above.

Preferred processes to be integrated include at least one of the reactions selected from isomerization, reforming, and disproportionation. In another preferred embodiment, at least two reactions selected from isomerization, reforming, and disproportionation are integrated. More preferred process include the integration of at least two of xylene isomerization, xylene reforming, and toluene disproportionation.

Preferred devices around which disparate processes are integrated include compressors.

One of skill in the art, in possession of the present disclosure, would recognize that a consequence of the presence of only a single compressor in the integrated process would result in the loss of control of the operation of each of the processes. As the two processes comprise a single compressor, it is no longer possible to control the processes independently. Thus, one may wish to compensate for this loss of control.

In an embodiment, the integrated process may comprise a controller to control process A and B such that the process B is controlled depending on the operation of process A. Process A and process B may be controlled such that process A operates at or close to its desired set point. This set point may correspond to the optimum production of the product of process A. Process B is then controlled such that the operation of process A is maintained at or close to its desired set point.

In a preferred embodiment, each of the processes A,B has a feedback line for recycling their respective intermediates. The compression step is located in the respective feedback lines of process A and process B. Each feedback line may comprise a feedback controller for controlling the flow rate of the intermediate. In this way, the processes A and B, can both be controlled even though they share the same compressor.

This is particularly important during start up of embodiments of the integrated process according to the invention. Complete closure of a feedback line allows process A to be started up and operated without interfering with process B. Once process A is fully operational, process B can be started up and the flow through the feedback line of process B is increased incrementally such that the set point of process A is not disturbed throughout the start up cycle. This then results in the integrated process being fully operational, operating at or near the set point of process A whereby process B is operated in accordance with this constraint.

The loss of control of the processes may further be compensated for by adjusting the operating parameters of the catalyst system(s), if used, in the plural processes (A, B, etc). In preferred embodiments, the two or more catalyst systems are selected to have similar operating pressures and similar recycle gas purity requirements. The present inventors have also determined that attention needs to paid to the contaminants in each of the respective catalysts systems to determine the impact on the other catalysts in the integrated systems.

In a further embodiment of the invention, there is provided a method of integrating a process A for producing a product PA and a process B for producing a product PB to form an integrated process I for converting one or more feeds into multiple products, each process A,B comprising a common intermediate process step, wherein a single device is provided for conducting the common intermediate process step. In an embodiment, each process A,B may comprise the step of compressing the respective intermediates.

In embodiments, the common intermediate process is conducted in a single device. The respective intermediates in the common intermediate step may comprise at least one common intermediate component. The common intermediate step may comprise, by way of example, compression of intermediates, such as hydrogen.

In a particular embodiment, the intermediates may consist of hydrogen. This allows for the advantageous toluene disproportionation and xylene isomerization process to be combined in a single, integrated process in which a single compressor is used. This considerably reduces the energy required to run the overall integrated process and reduces overall capital and running costs.

Examples

The invention may be better understood, and additional benefits to be obtained thereby realized, by reference to the following description of the figures, by way of examples. These examples should be taken only as illustrative of the invention rather than limiting, and one of ordinary skill in the art in possession of the present disclosure would understand that numerous other applications are possible other than those specifically enumerated herein

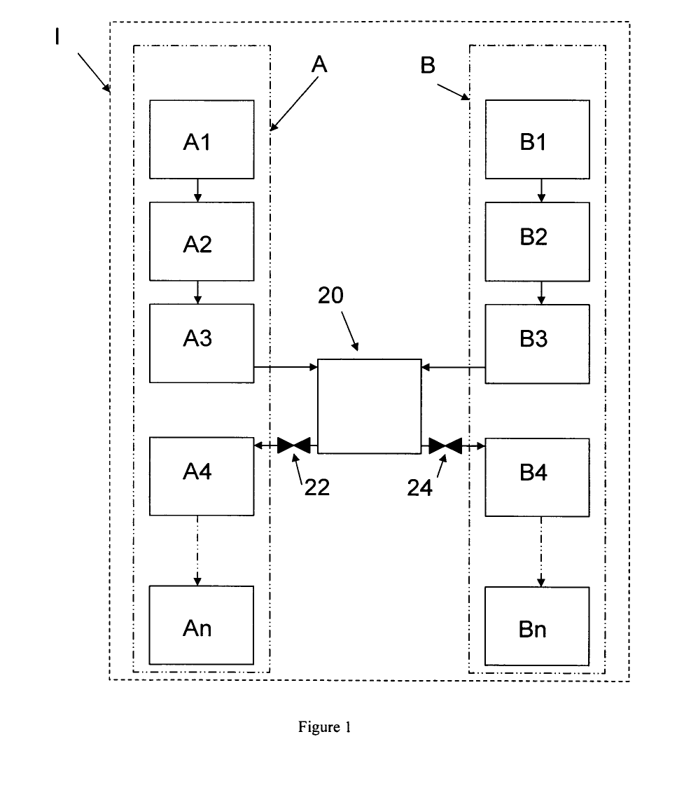

FIG. 1 shows an integrated process for converting one or more feeds into multiple products. The process comprises a process A for producing a product PA and a process B for producing product PB, each process comprising multiple steps A1 to An and B1 to Bn respectively. Each process also comprises a common intermediate process step 20, wherein a single device is provided for conducting the common intermediate process step 20.

The integrated process I comprises controllers 22, 24 for controlling the output of the common intermediate step 20. This allows each of the process A,B to be independently controlled.

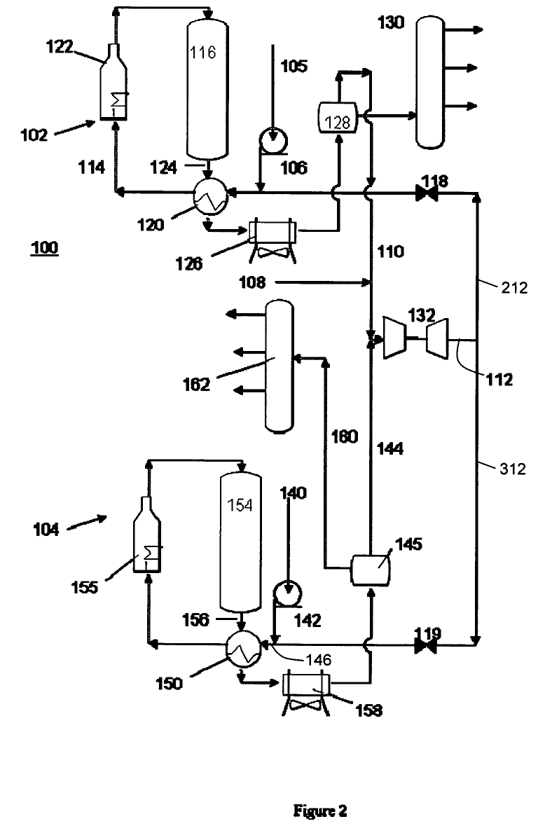

FIG. 2 shows a particular embodiment of the process of the invention. In this integrated process 100, the first process 102 is a toluene disproportionation process and the second process 104 is a xylene hydroforming or xylene isomerization process. Each of the processes 102, 104 comprises a reactor, 116, 154, heat exchangers, 120, 150, furnaces, 122, 155, separators 128, 145, distillation columns 130, 154, and a single, common compressor 132. We will now describe the integrated process in more detail.

In the disproportionation process 102, a liquid toluene feed 105 is pressurized by a feed pump 106 to the desired pressure which is required for the disproportionation reaction. Make-up hydrogen gas 108 is combined with recycle gas 110 to form the total recycle gas 112. Alternatively, make-up hydrogen gas may be provided to the discharge side of the compressor 132 in line 112. The combined feed 106 and recycle gas 212 form a feed stream 114 which has the desired target hydrogen:hydrocarbon molar ratio for the disproportionation reaction.

In the recycle gas 112, the hydrogen purity is adjusted to the minimum hydrogen partial pressure requirements. The resulting make up hydrogen 108 is determined and added to the process at the suction end of the compressor 132.

In FIG. 2, H₂ makeup 108 is shown on the suction side of the compressor but it could be on either side (suction or discharge). A hydrogen purge is also not shown in FIG. 2, but it can be present on one or more of: either side (suction or discharge) of the compressor or it could be on the recycle gas to either unit

The combined feed is preheated and completely vaporized in the feed/effluent heat exchanger 120. The stream is superheated in the fired heater 122 to the target temperature of the reactor 116. In the reactor 116, the toluene feed is converted to benzene and xylenes. More specific details about toluene disproportionation, and catalysts useful therefor, may be found in U.S. Pat. No. 5,365,004.

The reactor effluent 124 is cooled and partially condensed against the reactor feed and the feed/effluent heat exchanger 120. The stream is further condensed in the effluent cooler and cooled in the separator 126 to a temperature of 46° C. The stream than enters the high pressure separator 128, where hydrogen rich vapor is separated from the light hydrocarbons and C6+ aromatics liquid. The remainder of the vapor from the separator 128 is compressed in the recycled gas compressor 132 up to reaction pressure. Dry make up hydrogen 108 of 90.2 vol. % is added and a portion of the vapor from the separator is purged to control the target hydrogen recycled gas purity and hydrogen:hydrocarbon molar ratio.

Liquid from the high pressure separator 128 is sent to the deheptanizer column 130. In the deheptanizer C5 light gasses are separated from the reaction products and an overhead liquid stream containing mainly benzene and unreacted toluene are separated from the C8+ product. The overhead vapor is partially condensed in the column overhead exchanges and sent to an overhead accumulator drum (not shown). Liquid from the deheptanizer accumulator is pumped as reflux to deheptanizer column 130 (having plural take-offs, not numbered). The deheptanizer column 130 bottoms stream (not shown), made up of essentially C8+ aromatics, is recycled back to xylene re-run columns which is where the stream is separated into mixed xylenes and heavy aromatics (not shown).

Devices 118 and 119 are controllers, separately known in the art per se, used to control the rate of the gas recycle.

In the isomerization process 104, the xylene feed 140 is pressurized by a pump 142 to the pressure which is required in the reaction section. Make-up hydrogen 108 is combined with recycle gas 144 and 110 to form the total recycle gas 112 or, alternatively, the make-up hydrogen gas is provided to the discharge side of the compressor 132. The combined liquid 140 and 312 gas stream form a total reactive feed stream 146 which has the required hydrogen to hydrocarbon molar ratio. The hydrogen purity is adjusted in the total recycle gas stream 112 to meet minimum hydrogen partial pressure requirements.

The combined feed 146 is heated and completely vaporized in the feed/effluent heat exchange 150. The stream is superheated in the fired heater 155 to the required target reactor inlet temperature depending on the stage of the operating cycle. The stream then enters the reactor 154, which converts ethyl benzene to benzene and ethylene and isomerizes the para-xylene depleted stream to an equilibrium xylene distribution. More specific details for xylene isomerization, and catalysts useful therefor, may be found in U.S. Pat. 5,516,956.

The reactor effluent 156 is cooled and partially condensed against the reactor feed 146 in the feed oblique effluent heat exchange 150. The stream 156 is further condensed in the effluent airfin cooler 158 and cooled to the separated temperature of 46° C. The stream then enters a high pressure separator 145, where hydrogen rich vapor 144 is separated from the light hydrocarbon and C6+ aromatics liquid. The remainder of the vapor from the separator 162 is compressed in the recycle gas compressor 132 to the reaction pressure. Dry makeup hydrogen 108 with a purity of 90.2% is added and a portion of the vapor from the separator is purged to control the target hydrogen recycle gas purity and hydrogen:hydrocarbon molar ratio (not shown).

Liquid 160 from the high pressure separator is sent to the deheptanizer column 162, having plural take-offs (not numbered). In the deheptanizer column, C5− slight gasses are separated from the reaction products and an overhead (not shown) liquid stream containing mainly benzene and toluene separated from the C8+ product. The overhead vapor is partially condensed in the column 162 overhead exchanger and optionally sent to an overhead accumulator drum (not shown). The non-condensable light gas is removed via the deheptanizer column 162 off-gas stream. Liquid from the deheptanizer accumulator may be pumped as reflux to the tower (not shown) and also as a net liquid product which contains mainly benzene and toluene (benzene/toluene cut). The deheptanizer column 162 bottoms stream (not shown), made up of essentially C8+ aromatics, is recycled back to a xylenes column which is where the stream is separated from the mixed xylenes and heavy aromatics (not shown).

There is thus disclosed an integrated isomerization and disproportionation process in which the make up hydrogen is pressurized in a single compression step. This greatly improves the overall energy efficiency of the processes and has the additional benefit of reducing the overall capital cost.

Numerous variations on the above would be readily apparent to one of ordinary skill in the art in possession of the present disclosure.

For instance, in a preferred embodiment, a system for reforming may be integrated either with the above system, so that three separate and distinct processes share a common device (hydrogen compressor 132) or a reforming system replaces either the isomerization process 104 or the toluene disproportionation process 102. A particularly preferred reforming process comprises xylene reforming, per se known in the prior art.

The above examples merely illustrate the invention. Other, common process steps may be combined to further integrate multiple processes.

In yet still more preferred embodiments, the integrated processes comprise toluene disproporationation and xylene isomerization and in yet a still more preferred embodiment the integrated processes consist of toluene disproporationation and xylene isomerization. In either case, it is preferred that one of the processes (the first process) operate around a set point (e.g., with respect to pressure, feed rates and other variables that are within the skill of the ordinary artisan, in possession of the present disclosure, to select) and the other processes (the second process) is then optimized to allow the first process to operate around the preselected set point.

In the case of toluene disproportionation, the preferred catalysts are molecular sieves having a Constraint Index from about 1 to about 12 and include intermediate pore zeolites. Zeolites which conform to the specified values of constraint index for intermediate pore zeolites include ZSM-5, ZSM-11, ZSM-5/ZSM-11 intermediate, ZSM-12, ZSM-22, ZSM-23, ZSM-35, ZSM-48, ZSM-50, and ZSM-57. Such zeolites are described, for example, in U.S. Pat. No. 3,702,886 and Re. No. 29,949, U.S. Pat. Nos. 3,709,979, 3,832,449, 4,046,859, 4,556,447, 4,076,842, 4,016,245, 4,229,424, 4,397,827, 4,640,849, 4,046,685, 3,308,069 and Re. 28,341, to which reference is made for the details of these zeolites.

In an embodiment, the zeolite, either incorporated with a binder or in unbound form, is impregnated at least twice, preferably between about two and about six times, with a selectivating agent. The selectivating agent comprises a compound or polymer containing a main group or transition metal, preferably silicon. In each phase of the selectivation treatment, the selectivating agent is deposited on the external surface of the catalyst by any suitable method. For example, a selectivating agent, such as a silicon compound, may be dissolved in a carrier, mixed with the catalyst and then dried by evaporation or vacuum distillation. This method is termed “impregnation”. The molecular sieve may be contacted with the silicon compound at a molecular sieve/silicon compound weight ratio of from about 100/1 to about 1/100. More details may be found in U.S. Pat. No. 5,365,004.

In a preferred embodiment, the alkylbenzene may be fed simultaneously with a second selectivating agent and hydrogen at reaction conditions until the desired p-dialkylbenzene selectivity, e.g., 90%, is attained, whereupon the co-feed of selectivating agent is discontinued. This co-feeding of selectivating agent with alkylbenzene is termed “trim-selectivation”. Reaction conditions for this in situ trim-selectivation step generally include a temperature of from about 350° C. to about 540° C. and a pressure of from about atmospheric to about 5000 psig. The reaction stream is fed to the system at a rate of from about 0.1 WHSV to about 20 WHSV. Hydrogen may be fed at a hydrogen to hydrocarbon molar ratio of from about 0.1 to about 20.

In the preferred embodiment integrated with a xylene isomerization process, the toluene disproportionation reaction is operated around a set point preferably selected so that the xylene isomerization operates under conversion conditions including a temperature of from about 400° F. (about 200° C.). to about 1,000° F. (about 535° C.), a pressure of from about 0 to about 1,000 psig, a weight hourly space velocity (WHSV) of between about 0.1 and about 200 hr⁻¹, a hydrogen to hydrocarbon molar ratio of between 0.5 and about 10. Preferably, the conversion conditions include a temperature of from about 750° F (about 400° C.) and about 900° F (about 480° C.), a pressure of from about 50 and about 400 psig, a WHSV of between about 3 and about 50 hr⁻¹, and a hydrogen to hydrocarbon molar ratio of between about 1 and about 5.

In more preferred embodiment, the catalyst system of the isomerization process comprises two catalysts. One of the catalysts, the first catalyst, is selective for ethylbenzene conversion while minimizing xylene loss. The other catalyst of the system, the second catalyst, isomerizes the xylenes to effect isomerization to the extent that the amount of para-xylene in the isomerization product is approximately equal to or greater than that at the thermal equilibrium of the xylene(s). In one embodiment of the process, the first catalyst will also show reduced activity for isomerization of the xylenes. Preferred examples for both the first and second catalysts include ZSM-5; ZSM-11, ZSM-12, ZSM-21, ZSM-22, ZSM-23, ZSM-35, ZSM-38 ZSM-48, ZSM-57, ZSM-58, and mixtures thereof. Specific catalysts systems are set forth in U.S. Pat. No. 5,516,956.

The meanings of terms used herein shall take their ordinary meaning in the art; reference shall be taken, in particular, to Handbook of Petroleum Refining Processes, Third Edition, Robert A. Meyers, Editor, McGraw-Hill (2004). All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. Trade names used herein are indicated by a ™ symbol or ® symbol, indicating that the names may be protected by certain trademark rights, e.g., they may be registered trademarks in various jurisdictions.

1. An integrated process comprising at least two chemical processes integrated around at least one common device, said integrated process characterized by having at least two separate and distinct feedstreams, two separate and distinct products, or a combination thereof, and said at least two chemical processes having a common intermediate step.

2. The integrated process according to claim 1, wherein said common intermediate process step comprises compression of a gas.

3. The integrated process according to claim 1, wherein said at least one common device is a compressor.

4. The integrated process according to claim 1, further characterized as comprising a first process A for producing a product PA, and a second process B, different from A, for producing a product PB, which may be the same or different from PA, each separate process, A and B, having a common intermediate process step and wherein said at least one common device is provided for conducting said common intermediate process step.

5. The integrated process according to claim 1, wherein said at least two chemical processes are selected from the group consisting of disproportionation, isomerization, and reforming.

6. The integrated process according to claim 1, wherein one of the at least two chemical processes comprises the disproportionation of toluene.

7. The integrated process according to claim 1, wherein one of the at least two chemical processes comprises isomerization of xylene.

8. The integrated process according to claim 1, wherein one of the at least two chemical processes comprises xylene reforming.

9. The integrated process according to claim 1, wherein said common intermediate process step is the compression of hydrogen.

10. The integrated process according to claim 1, wherein said common intermediate process step comprises compression of a gas, and subsequent recycling of the resulting compresses gas to at least one of said at least two chemical processes.

11. The integrated process according to claim 1, wherein the operating characteristics of a first of said at least two chemical processes are controlled around a preselected set point.

12. The integrated process according to claim 1, wherein said integrated process is characterized by a common product.

13. The integrated process according to claim 12, wherein said common product is xylene.

14. The integrated process according to claim 4, wherein process A comprises alkylbenzene disproportionation and process B comprises xylene isomerization.

15. The integrated process according to claim 14, wherein process A is characterized by a step comprising feeding said alkylbenzene simultaneously with a co-feed of selectivating agents and hydrogen at a temperature of from about 350° C. to about 540° C. and a pressure of from about atmospheric to about 5000 psig at a rate of from about 0.1 WHSV to about 20 WHSV, wherein the hydrogen gas to hydrocarbon molar ratio is from about 0.1 to about 20, and process B is characterized by xylene isomerization conversion conditions including a temperature of from about 200° C. to about 535° C., a WHSV of between about 0.1 and about 200 hr⁻¹, and a hydrogen to hydrocarbon molar ratio of between 0.5 and about 10, and wherein both process A and process B are further characterized as comprising a step of hydrogen gas compression in a common compressor and wherein the discharge from said common compressor is recycled to both process A and process B.

16. The process according to claim 15, wherein the catalyst system of process A comprises a zeolite selected from ZSM-5, ZSM-11, ZSM-12, ZSM-22, ZSM-23, ZSM-35, ZSM-48, ZSM-50, ZSM-57, and mixtures thereof, said zeolite treated with at least one selectivating agent, and the catalyst system of process B comprises a zeolite selected from ZSM-5; ZSM-11, ZSM-12, ZSM-21, ZSM-22, ZSM-23, ZSM-35, ZSM-38 ZSM-48, ZSM-57, ZSM-58, and mixtures thereof, said zeolite treated with at least one selectivating agent.
